(19) United States
(12) Patent Application Publication
(10) Pub. No.: US 2006/0058261 A1
(43) Pub. Date: Mar. 16, 2006

(54) Chitin derivatives for hyperlipidemia

(76) Inventor: Andre Aube, St-Sauveur (CA)

Correspondence Address: KNOBBE MARTENS OLSON & BEAR LLP, 2040 MAIN STREET, FOURTEENTH FLOOR, IRVINE, CA, 92614, US

(21) Appl. No.: 11/218,348

(22) Filed: Sep. 1, 2005

Related U.S. Application Data
(60) Provisional application No. 60/609,830, filed on Sep. 15, 2004.

(51) Int. Cl. A61K 31/722

(52) U.S. Cl. 514/55

(57) ABSTRACT

The preferred embodiments relate to chitin derivatives for prevention or treatment of hyperlipidemia, such as hypercholesterolemia and the resultant atherosclerosis in a mammal. The preferred embodiments are useful for reducing serum cholesterol, and/or cholesteryl ester, triglycerides, phospholipids and fatty acids in a mammal.

### CROSS REFERENCE TO RELATED APPLICATIONS

This application claims the benefit under 35 U.S.C. § 119 (e) of U.S. Provisional Application No. 60/609,830, filed Sep. 15, 2004.

### BACKGROUND OF THE INVENTION

1. Field of the Invention

The present invention relates to the field of therapeutic agents useful in lowering cholesterol (particularly low-density cholesterol) and/or, cholesteryl esters, triglycerides, phospholipids, and fatty acids in a mammal, such as a human. More particularly, the invention relates to compositions comprising chitin derivatives.

2. Description of the Related Art

It is well-known that hyperlipidemic conditions associated with elevated concentrations of total cholesterol and low-density lipoprotein (LDL) cholesterol are major risk factors for cardiovascular diseases, such as atherosclerosis. Numerous studies have demonstrated that a low plasma concentration of high density lipoprotein (HDL) cholesterol (good cholesterol) is a powerful risk factor for the development of atherosclerosis (Barter and Rye, Atherosclerosis, 121, 1-12 (1996). HDL is one of the major classes of lipoproteins that function in the transport of lipids through the blood. The major lipids found associated with HDL include cholesterol, cholesteryl esters, triglycerides, phospholipids, and fatty acids. The other classes of lipoproteins found in the blood are low density lipoprotein (LDL), intermediate density lipoprotein (IDL), and very low density lipoprotein (VLDL). Since low levels of HDL cholesterol increase the risk of atherosclerosis, methods for elevating plasma HDL cholesterol would be therapeutically beneficial for the treatment of cardiovascular diseases, such as atherosclerosis. Cardiovascular diseases include, but are not limited to, coronary heart disease, peripheral vascular disease, and stroke.

One therapeutic approach to hyperlipidemic conditions has been the reduction of total cholesterol. Known use is made of the understanding that HMG CoA reductase catalyzes the rate-limiting step in the biosynthesis of cholesterol (The Pharmacological Basis of Therapeutics, 9th ed., J. G. Hardman and L. E. Limberd, ed., McGraw-Hill, Inc., New York, pp. 884-888 (1996)). HMG CoA reductase inhibitors (including the class of therapeutics commonly called “statins”) reduce blood serum levels of LDL cholesterol by competitive inhibition of this biosynthetic step (M. S. Brown, et al., J. Biol. Chem. 253, 1121-28 (1978)). Several statins have been developed or commercialized throughout the world. Atorvastatin calcium sold in North America under the brand Lipitor® is a potent reductase inhibitor. It is described in European Patent 409,281.

Warnings of side effects from use of HMG CoA reductase inhibitors include liver dysfunction, skeletal muscle myopathy, rhabdomyolysis, and acute renal failure. Some of these effects are exacerbated when HMG CoA reductase inhibitors are taken in greater doses. For example, a patient treated with 10 mg/day of Lipitor® may notice mild side effects. These side effects may greatly increase by simply raising the daily dose to 20 mg/day.

Furthermore, it has been shown that patients with well-controlled lipid profiles when treated at 10 mg/day may experience a return to elevated lipid profiles and require a dosage increase.

Thus, although there is a variety of hypercholesterolemia therapies, there is a continuing need and a continuing search in this field of art for alternative therapies.

### SUMMARY OF THE INVENTION

The preferred embodiments improve efforts for preventing and/or treating hyperlipidemia, such as by reducing serum cholesterol, by providing a composition comprising chitin derivatives.

An embodiment provides a pharmaceutical composition comprising a chitin derivative having a molecular weight of about 30 to about 60 kDa.

An embodiment provides a method for preventing or treating a hyperlipidemia or hyperlipidemia-associated condition comprising administering a pharmaceutical composition comprising a chitin derivative having a molecular weight of about 30 to about 60 kDa.

The use of a chitosan derivative of the preferred embodiments in a pharmaceutical composition is especially advantageous in that the chitosan derivative (salt) can remain stable in the composition for a prolonged period. Natural chitosan remains stable for a period of a few weeks whereas the chitosan derivatives of the preferred embodiments will remain stable in the composition for at least 2 years.

The chitosan derivative composition of the preferred embodiments further has the advantage of providing a low side effect alternative therapy against hyperlipidemia.

### DETAILED DESCRIPTION OF THE PREFERRED EMBODIMENT

It has been found that chitin derivatives having a molecular weight of about 30 to about 60 kDa can lower cholesterol levels. In a preferred embodiment, the chitin derivative has a molecular weight of about 30 kDa. In another preferred embodiment, the chitin derivative has a molecular weight of about 40 to about 50 kDa.

As used herein, “chitin” refers to a polymer formed primarily of repeating units of β (1-4) 2-acetamido-2-deoxy-D-glucose (or N-acetylglucosamine). Not every unit of naturally occurring chitin is acetylated, with about 16% deacetylation.

As used herein, “chitosan” refers to chitin that has been partially or fully deacetylated. Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

As used herein, “derivative” refers to a chemical composition derived from another substance either directly or by modification or partial substitution.

As used herein, the terms “chitin derivative” and “chitosan derivative” can be used interchangeably and can encompass each other herein. The term “chitin derivative” is also understood herein to encompass a chitosan salt formed from any chitosan molecule associated with a negatively charged anion. A series of anions has been used for that purpose. For example, anions can be derived from inorganic acids. Preferred inorganic anions include, but are not limited to, sulfuric acid (sulfate), phosphoric acid (phosphate), hydrochloric acid (chloride). Anion can also be derived from organic acids. Preferred organic anions include, but are not limited to, malic acid (malate), tartaric acid (tartrate), citric acid (citrate) and lactic acid (lactate).

Chitosan is a naturally-occurring biopolymer that can also be obtained by partial or complete deacetylation of chitin that is the major component of the exoskeleton of shellfishes and insects. Chitosan is therefore a linear polymer composed of monomers of N-acetyl-2-amino-β-D-glucose and 2-amino-β-D-glucose. The presence of the primary amino groups of the 2-amino-β-D-glucose (D-glucosamine) units confers to chitosan its polycationic (positively charged) character that is neutralized by accompanying negatively charged anions. A series of anions has been used for that purpose. For example, anions derived from inorganic acids such as, but not limited to, sulfuric acid (sulfate), phosphoric acid (phosphate), hydrochloric acid (chloride) and a mixture thereof and organic acids, such as, but not limited to, malic acid (malate), tartaric acid (tartrate), citric acid (citrate), lactic acid (lactate), acetic acid (acetate), formic acid (formate), glycolic acid (glycolate), oxalic acid, succinic acid, ascorbic acid, maleic acid, acrylic acid, gluconic acid, glutamic acid, propionic acid and a mixture thereof have been reported as salts of chitosan.

While there exists many extraction methods of the chitin from the crustacean shells, the principles of chitin extraction are relatively simple. In a certain treatment, the proteins are removed in a dilute solution of sodium hydroxide (such as about 1-10%) at high temperature (such as about 85-100° C.). Shells are then demineralized to remove calcium carbonate. This can be done by treating in a dilute solution of hydrochloric acid (1-10%) at room temperature. Depending on the severity of these treatments such as temperature, duration, concentration of the chemicals, concentration and size of the crushed shells, the physico-chemical characteristics of the extracted chitin can vary. For instance, three characteristics of the chitin, such as the degree of polymerization, acetylation, and purity, can be affected. Shell also contains lipids and pigments. Therefore, a decolorizing step is sometimes needed to obtain a white chitin. This can be done by soaking in organic solvents or in a very dilute solution of sodium hypochlorite. Again, these treatments can influence the characteristics of the chitin molecule.

Chitin can be deacetylated partially or totally. Such a deacetylated polymer is called chitosan. Chitosan compounds in a range of up to and exceeding 1×10⁶ molecular weight are derived commercially from chitin. In nature, chitosan is present in cell walls of Zygomycetes, a group of phytopathogenic fungi. Because of its significant content of free amino groups, chitosan has a markedly cationic character and has a positive charge at most pHs. Chitosan can be obtained by a process disclosed in Canadian Patent 2,085,292, the disclosure of which is incorporated herein by reference.

Chitin derivatives may be produced by the process described in WO 2005/066213-A1, where the chitosan is salted out with a salting-out salt such as sulfates, phosphates, citrates, nitrates, malates, tartrates, succinates, propionates, lactates and hydrogen phosphates. More preferably, these salting-out salts may be selected from the group consisting of: ammonium or sodium sulfate; sodium or potassium phosphates; sodium or potassium citrate; sodium tartrate; sodium malate; sodium nitrate; sodium lactate; sodium malonate; sodium succenate; sodium acetate; sodium propionate. Thus, the preferred embodiments includes any chitosan derivative obtained by any of the above-mentioned salts.

As an example, the citrate salt of chitosan can be illustrated as follows:

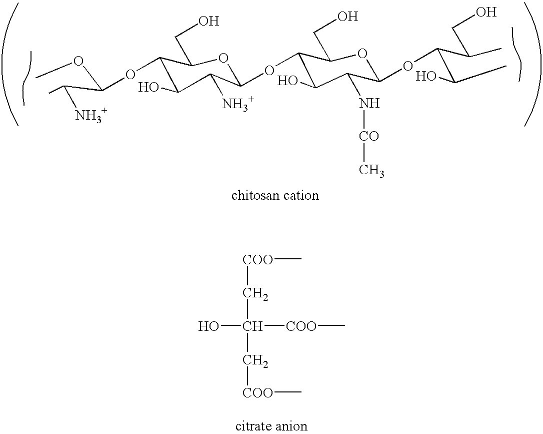

An approach for addressing hyperlipidemia is a use of chitin derivatives.

In a mechanism of action, chitin derivatives, in particular chitosan, can contain free amine groups which can attach themselves to lipids, such as cholesterol, via ionic bonds while in the intestinal tractus, forming an indissociable complex which is eventually excreted. Chitin derivatives therefore can prevent lipids, such as cholesterol, from ever entering the bloodstream and adding to the total cholesterol content. Also, in reaction, the liver eliminates more cholesterol by using biliary acids. Therefore, there is elimination of both food cholesterol and that of biliary acids rich in cholesterol.

Molecular Weight

Chitin derivatives have many potential applications depending on their molecular weight. The molecular weight can be measured by any of a number of well known techniques, including, without limitation, by SDS-PAGE or mass spectrometry. These techniques can yield various types of molecular weights, including without limitation, apparent molecular weight, a weight average molecular weight, or a number average molecular weight. An average high molecular weight chitin derivative is about 650 kDa. Some applications are typical of medium or low molecular weight chitin derivatives, ranging typically about 2-500 kDa. These applications include its use as an antifungal agent; a seed coating for improving crop yield; an elicitor of anti-pathogenic natural reactions in plants; a hypocholesterolemic agent in animals; an accelerator of lactic acid bacteria breeding; and a moisture-retaining agent for lotions, hair tonics and other cosmetics.

The molecular weight of chitin derivatives is a feature that is particular to a certain application. The molecular weight of the native chitin has been reported to be as high as many million Daltons. However, chemical treatment tends to bring down the molecular weight of the chitin derivative, ranging from 100 KDa to 1500 KDa. Further treatment of the chitin derivative can lower the molecular weight even more. Low molecular weight could be produced by different ways including enzymatic or chemical methods. When the chain becomes shorter, the chitin derivative can be dissolved directly in water without the need of an acid. This is particularly useful for specific applications, such as in cosmetics or in medicine. Molecular weight of the chitin derivative can be measured by analytical methods, such as gel permeation chromatography, light scattering, or viscometry. Because of simplicity, viscometry is the most commonly used method.

In the preferred embodiments, the chitin derivative has molecular weight of about 30 to about 60 kDa. Preferably, the chitin derivative has molecular weight of about 30 kDa. Preferably, the chitin derivative has molecular weight of about 27.5, 28, 28.5, 29, 29.5, 30, 30.5, 31, 31.5, 32, or 32.5 kDa.

In another embodiment, the chitin derivative preferably has molecular weight of about 40 to about 50 kDa. More preferably, the chitin derivative has molecular weight of about 40, 41, 42, 43, 44, 45, 46, 47, 48, 49, or 50 kDa.

Deacetylation

Chitin can be deacetylated partially or totally. Naturally occurring chitin is acetylated, with about 16% deacetylation. Chitosan refers to chitin that has been partially or fully deacetylated. Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

Since chitosan is made by deacetylation of chitin, the term degree of deacetylation (DAC) can be used to characterize chitosan. This value gives the proportion of monomeric units of which the acetylic groups that have been removed, indicating the proportion of free amino groups (reactive after dissolution in weak acid) on the polymer. DAC could vary from about 70 to about 100%, depending on the manufacturing method used. This parameter indicates the cationic charge of the molecule after dissolution in a weak acid. There are many methods of DAC measurements, such as UV and infrared spectroscopy, acid-base titration, nuclear magnetic resonance, dye absorption, and the like. Since there are no official standard methods, numbers tend to be different for different methods. In high value product, NMR can give a precise DAC number. However, titration or dye adsorption can serve as a quick and convenient method and yield similar results as NMR.

Chitin deacetylation towards chitosan can be obtained by various methods. The most used method is that of alkaline treatment (Horowitz, S. T. et al., 1957). With this method, around 80% of deacetylation can be achieved without significant decrease of molecular weight. A more intense deacetylation cannot be obtained by this method without a simultaneous uncontrolled decrease of the degree of polymerization. A more promising method is deacetylation by a thermo-mechano-chemical treatment (Pelletier et al., 1990). This method allows a more careful control of the various characteristics of the final product (average degree of polymerization and of deacetylation). Finally, a third method (Domard and Rinaudo, 1983) allows obtainment of a totally deacetylated product.

In a certain deacetylation protocol, when chitin is heated in a basic solution, such as a strong solution of sodium hydroxide (such as >about 40%) at high temperature (such as about 90-120° C.), chitosan is formed by deacetylation. This treatment can remove acetylic grouping on the amine radicals to a product (chitosan) that could be dissolved. It is said that at least 65% of the acetylic groups should be removed on each monomeric chitin to obtain the ability of being put in solution. The degree of deacetylation will vary according to the treatment conditions, such as duration, the temperature, and the concentration of the basic solution.

In the preferred embodiments, the chitin derivative has a deacetylation higher than about 80%. Preferably, the chitin derivative has a deacetylation higher than about 89%. More preferably, the chitin derivative has a deacetylation higher than about 89, 90, 91, 92, 93, 94, 95, 96, 97, 98, 99, or 100%. In a chitin derivative that has been deacetylated about 100%, the advantage being the chitin derivative forms a relatively homogeneous composition.

Pharmaceutical Compositions

The compounds useful in the preferred embodiments can be presented with an acceptable carrier in the form of a pharmaceutical composition. The carrier is acceptable in the sense of being compatible with the other ingredients of the composition and is not be deleterious to the recipient. The carrier can be a solid or a liquid, or both, and is preferably formulated with the compound as a unit-dose composition, for example, a capsule or tablet, which can contain from about 0.05% to about 95% by weight of the active compound. Examples of suitable carriers, diluents, and excipients include, but are not limited to, lactose, dextrose, sucrose, sorbitol, mannitol, starches, gum acacia, alginates, tragacanth, gelatin, calcium silicate, cellulose, magnesium carbonate, or a phospholipid with which the polymer can form a micelle. Other pharmacologically active substances can also be present. The pharmaceutical compositions of the preferred embodiments can be prepared by any of the well-known techniques of pharmacy, comprising admixing the components.

As previously mentioned, the use of chitosan derivative allows the production of a pharmaceutical composition that may have a prolonged shelf life compared to the use of natural chitosan. It is a well-established fact that uncharged primary amines are more susceptible to oxidation. In contrast, the corresponding salts confer increased stability due to the fact of protonation of the lone pair of electrons of the nitrogen atom. This basic principle also applies to the chitosan polymer due to the presence of the large number of primary amino groups (D-glucosamine units) composing its backbone. In this respect, the salts of chitosan described above will confer stability over long periods of storage.

Whereas a number of salts of chitosan can be used to increase its stability under storage conditions, the selection of the nature of the chitosan salt may be dictated by the intended purpose of its use. For instance, chitosan salts that are compatible with food offer a definitive advantage for their uses as a diet supplement or for other purposes related to human or animal applications. The citrate salt of chitosan has been found to fulfill this requirement in two ways. First, it is a food-compatible salt and second, it confers to the natural chitosan molecule an extended shelf life.

In practicing the methods of the preferred embodiments, administration of the preferred embodiments may be accomplished by oral route or by intravenous, intramuscular, subcutaneous injections, or a combination thereof.

For oral administration, preferred embodiments can be in the form of, for example, but not limited to, a tablet, a capsule, a suspension, powders (e.g., for sprinkling on food), or liquid. Capsules, tablets, liquid, or powders, and the like can be prepared by conventional methods well-known in the art. The compounds are preferably made in the form of a dosage unit containing a specified amount of the compound. In one embodiment, the composition is in the form of a sustained release formulation.

When the chitosan derivative in the form of powder is obtained, encapsulation proceeds. If the powder is composed of multiple lots, a “tri bender” is thus used to provide a uniform admixture of the various lots. In some cases, the powder granulometry is not uniform and a sieving of the powder is therefore necessary in order to obtain the required granulometry for the type of encapsulation equipment that is used. Such sieving of the powder is accomplished either by coring or gravity. During encapsulation, some capsules are sampled and weighed to provide a uniform filling. Capsules of size 00 are used to hold 800 mg of chitosamine derivative per capsule. Capsules of size 00 or 01 may also be used for lower chitosamine derivative doses, for example 400 mg to 600 mg.

A preferred total daily dose of about 400 mg to about 4.8 grams per day and preferably between about 800 mg and 3.2 grams per day may generally be appropriate. More preferably, the total daily dose may range from 1.6 grams to 2.4 grams per day. The chitin derivative will preferably be taken three times a day, or preferably twice a day and more preferably once a day in a sustained release system (mode). The chitin derivative will preferably be taken with meals.

The daily doses for the preferred embodiments can be administered to the patient in a single dose, or in proportionate multiple subdoses. Subdoses can be administered about 2 to about 3 times per day. In a single dosage regimen, doses can be in a sustained release form that is effective to obtain desired results.

The dosage regimen to treat hyperlipidemia and hyperlipidemia-associated conditions, and reduce plasma cholesterol with the preferred embodiments is selected in accordance with a variety of factors. These factors include, but are not limited to, the type, age, weight, sex, diet, and medical condition of the patient, the severity of the disease, the route of administration, pharmacological consideration, such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from a hyperlipidemic condition, such as, but not limited to, hypercholesterolemia and atherosclerosis, can begin with the dosages indicated above. Treatment should generally be continued as necessary over a period of several weeks to several months or years until the condition has been controlled or eliminated. Patients undergoing treatment with the compounds or compositions disclosed herein can be routinely monitored by, for example, measuring serum LDL and total cholesterol levels by any of the methods well-known in the art, to determine the effectiveness of the therapy.

Prevention and Treatment of Conditions

The preferred embodiments can be used to prevent, give relief from, or ameliorate a disease condition having hyperlipidemia as an element of a disease, such as atherosclerosis or coronary heart disease, or to protect against or treat further high cholesterol plasma or blood levels with the compounds and/or compositions of the preferred embodiments. The pharmaceutical composition of the preferred embodiments thus prevents, gives relief from or ameliorates the above-mentioned hyperlipidemia-associated diseases by increasing the level of HDL, decreasing the level of LDL and/or decreasing the level of total cholesterol by increasing the ratio of HDL/LDL. Hyperlipidemia is an elevation of lipids (fats) in the bloodstream. These lipids include cholesterol (including HDL, LDL), cholesterol esters (compounds), phospholipids, triglycerides, and fatty acids. These lipids are transported in the blood as part of large molecules called lipoproteins.

Adverse effects of hyperlipidemia include atherosclerosis and coronary heart disease. Atherosclerosis is a disease characterized by the deposition of lipids, including cholesterol, in the arterial vessel wall, resulting in a narrowing of the vessel passages and ultimately hardening the vascular system. The primary cause of coronary heart disease (CHD) is atherosclerosis. CHD occurs when the arteries that supply blood to the heart muscle (coronary arteries) become hardened and narrowed. As a result of CHD, there could be angina or heart attack. Over time, CHD can weaken your heart muscle and contribute to heart failure or arrhythmias.

Hypercholesterolemia is also linked with cardiovascular disease. Cardiovascular disease refers to diseases of the heart and diseases of the blood vessel system (arteries, capillaries, veins) within a person's entire body, such as the brain, legs, and lungs. Cardiovascular diseases include, but are not limited to, coronary heart disease, peripheral vascular disease, and stroke.

Accordingly, the preferred embodiments may be used in preventing or treating hyperlipidemia and conditions associated with hyperlipidemia, such as hypercholesterolemia, atherosclerosis, coronary heart disease, and cardiovascular disease.

The disclosure below is of specific examples setting forth preferred methods. These examples are not intended to limit the scope, but rather to exemplify preferred embodiments.

### EXAMPLE 1

In a preferred embodiment, a chitin derivative has a molecular weight of about 30 kDa and is deacetylated at least 80%. In a preferred embodiment, the chitin derivative has a molecular weight of about 30 kDa and is deacetylated at least about 93% and is sold in Canada under the trademark Libracol®. In a preferred embodiment, there is provided a gelatin capsule containing about 800 mg of a chitin derivative.

Those skilled in the art will know, or be able to ascertain, using no more than routine experimentation, many equivalents to the specific embodiments of the invention described herein. These and all other equivalents are intended to be encompassed by the following claims.

What is claimed is:

1. A pharmaceutical composition comprising a chitin derivative having a molecular weight of about 30 to about 60 kDa.

2. The pharmaceutical composition of claim 1, wherein the chitin derivative has a molecular weight of about 30 kDa.

3. The pharmaceutical composition of claim 1, wherein the chitin derivative is further deacetylated by chemical or biological treatment.

4. The pharmaceutical composition according to claim 3, wherein the chitin derivative is deacetylated at least about 80%.

5. The pharmaceutical composition of claim 4, wherein the chitin derivative is deacetylated at least about 93%.

6. The pharmaceutical composition of claim 1, further comprising a pharmaceutically acceptable carrier.

7. A method for preventing or treating a hyperlipidemia or hyperlipidemia-associated condition comprising administering a pharmaceutical composition of claim 1.

8. The method of claim 7, wherein the hyperlipidemia-associated condition is selected from the group consisting of hypercholesterolemia, atherosclerosis, coronary heart disease, and cardiovascular disease.

9. The method of claim 7, wherein the pharmaceutical composition is administered in an amount ranging from 400 mg to 4.8 grams per day.

10. The method of claim 9, wherein the pharmaceutical composition is administered in an amount ranging from 1.6 grams to 2.4 grams per day.

11. A method for preventing or treating a hyperlipidemia or hyperlipidemia-associated condition comprising administering a pharmaceutical composition of claim 2.

12. The method of claim 11, wherein the hyperlipidemia-associated condition is selected from the group consisting of hypercholesterolemia, atherosclerosis, coronary heart disease, and cardiovascular disease.

13. The method of claim 11, wherein the pharmaceutical composition is administered in an amount ranging from 400 mg to 4.8 grams per day.

14. The method of claim 13, wherein the pharmaceutical composition is administered in an amount ranging from 1.6 grams to 2.4 grams per day.
